(19) United States
(12) Patent Application Publication
(10) Pub. No.: US 2014/0234570 A1
(43) Pub. Date: Aug. 21, 2014

(54) METHOD OF PRODUCING FLAT COCOON PRODUCT AND FLAT COCOON PRODUCT

(71) Applicant: Akihiko IZUKURA, Kyoto (JP)

(72) Inventor: Akihiko IZUKURA, Kyoto (JP)

(21) Appl. No.: 14/185,201

(22) Filed: Feb. 20, 2014

(30) Foreign Application Priority Data
Feb. 21, 2013 (JP) 2013-032595

(51) Int. Cl. C07K 14/435; B29C 43/18; B29C 43/00

(52) U.S. Cl. CPC C07K 14/43586; B29C 43/003; B29C 43/18
USPC 428/76; 530/353; 264/325; 264/271.1

(57) ABSTRACT

Provided is a three-dimensional flat cocoon product which is lightweight, has a favorable design and yields a highly uniform flat cocoon throughout the entire area thereof. The method of producing a flat cocoon product includes: expanding an expandable/contractible three-dimensional molding die; molding a flat cocoon by causing silkworms to crawl on an outer surface of the three-dimensional molding die and spin a cocoon; and contracting the three-dimensional molding die after the molding of the flat cocoon and extracting the contracted three-dimensional molding die from the flat cocoon.

### TECHNICAL FIELD

The present invention relates to a method of producing a flat cocoon product and a flat cocoon product.

### BACKGROUND ART

It is known that a flat cocoon is formed by causing silkworms to crawl on a mold frame which is formed in a planar shape and spin a cocoon in a flat fabric shape rather than spinning a cocoon ball, and it is also known that a three-dimensional flat cocoon product can be produced by applying the foregoing flat cocoon technique.
Patent Document: Japanese Patent Application Publication No. 2009-24314.

Patent Document describes the production of a hat as a three-dimensional flat cocoon product in which the front face side thereof is protruding and the back face side thereof is recessed by causing silkworms to crawl on the front face side and the back face side of a mold frame which is formed in a hat shape and spin a cocoon.

### DISCLOSURE OF THE INVENTION

Nevertheless, with the hat produced with the conventional technology of Patent Document the mold frame will remain inside the hat since the cocoon is spun on both the front and back face sides of the mold frame and, therefore, there is a problem in that the weight of the hat will increase due to the mold frame and this weight causes the wear comfort of the hat to deteriorate.

A three-dimensional flat cocoon product in which a mold frame remains therein as described above is not limited to the foregoing hat, and also applies to a lamp shade or a lantern, and there is a problem in that, the larger the item, the greater the influence of the weight of the mold frame.

With the three-dimensional flat cocoon product described above the mold frame can be seen through the surface of the flat cocoon product and the see-through mold frame sometimes constitutes a part of the design depending on the flat cocoon product. Nevertheless, the configuration of a mold frame which enables silkworms to spin a flat cocoon while using that mold frame as a part of the design of the three-dimensional flat cocoon product and inhibiting the increase in weight of the flat cocoon product poses a problem of low design flexibility and there is a problem in that the design becomes limited.

The present invention has been made to address the foregoing problems. Specifically, an object of this invention is to enable weight saving and flexible designability of a three-dimensional flat cocoon product as well as the reliable spinning of a flat cocoon by silkworms and consequently provide a three-dimensional flat cocoon product which is lightweight, has a satisfactory design and yields a highly uniform flat cocoon throughout the entire area thereof.

In order to achieve the foregoing object the present invention comprises at least the following configuration.

A method of producing a flat cocoon product comprising: expanding an expandable/contractible three-dimensional molding die; molding a flat cocoon by causing silkworms to crawl on an outer surface of the three-dimensional molding die and spin a cocoon; and contracting the three-dimensional molding die after the molding of the flat cocoon and extracting the contracted three-dimensional molding die from the flat cocoon.

A flat cocoon product that is produced with the foregoing production method.

The present invention having the foregoing features yields the following effects. Specifically, since a flat cocoon is molded on the outer surface of the expanded three-dimensional molding die and the three-dimensional molding die is contracted and extracted after the molding of the flat cocoon, it is possible to enable weight saving and flexible designability of a three-dimensional flat cocoon product as well as the reliable spinning of a flat cocoon by silkworms. Consequently, it is possible to provide a three-dimensional flat cocoon product which is lightweight, has a satisfactory design and yields a highly uniform flat cocoon throughout the entire area thereof.

### BRIEF DESCRIPTION OF THE DRAWINGS

FIG. 1 is a perspective view showing an example of a figurine of an embodiment according to the present invention;

FIG. 2 is a cross section of line (2)-(2) in FIG. 1;

FIGS. 3(a) to 3(c) are process diagrams showing an example of the method of producing a figurine of an embodiment according to the present invention; and

FIGS. 4(d) to 4(f) are process diagrams showing an example of the method of producing a figurine continuing from FIGS. 3(a) to 3(c).

### DETAILED DESCRIPTION

Preferably, the present invention further comprises a step of arranging a decorative member on the surface of the three-dimensional molding die in an expanded state, wherein the contracted three-dimensional molding die is extracted in a state where the decorative member is left on an inner face of the molded flat cocoon.

The expandable/contractible three-dimensional molding die explained below is, for example, a rubber balloon or a so-called beach ball which can be expanded by pumping air therein and contracted by releasing air therefrom or a bag or the like which can be expanded by filling a padding therein and contracted by removing the padding therefrom.

Moreover, as the shape of a three-dimensional molding die, for example, used may be a spherical shape, a cube shape, a conical shape, a hat shape, a human shape (whole body, upper body, lower body) or the like.

Moreover, when the three-dimensional molding die is a bag or the like which can be expanded by filling a padding therein as described above, by using a bag and a padding made of a deformable material such as a paper material or a synthetic resin material, the three-dimensional molding die can be deformed into the shapes described above, namely, a spherical shape, a cube shape, a conical shape, a hat shape, a human shape (whole body, upper body, lower body) or the like.

The decorative member explained below is, for example, a member that can be seen through from the inner side of the molded flat cocoon. For example, the decorative member may be a colored string or band-like member, a small light bulb (midget lamp or the like) provided on a bendable cable, a light-emitting diode element (LED) provided on a similarly bendable, thin band-shaped circuit, optical fibre, electroluminescence (EL) sheet, EL fibre, EL cord or the like. As a result of arranging this kind of decorative member by wrapping it around the three-dimensional molding die, it is possible to decorate the flat cocoon in a manner in which the decorative member can be seen through from the outside of the flat cocoon.

The flat cocoon product explained below is, for example, a decor such as a lamp shade or a figurine, a hat, clothing, a bag, a pouch or the like.

The flat cocoon explained below refers to a cocoon that is molded in a three-dimensional shape as the external form by causing silkworms to crawl on the surface of the three-dimensional molding die and spin a cocoon rather than causing the silkworms to spin a cocoon ball, and it does not refer to a cocoon that is molded in a shape of a single sheet of flat fabric.

The method of producing a flat cocoon and the flat cocoon product of an embodiment according to the present invention are now explained with reference to the drawings.

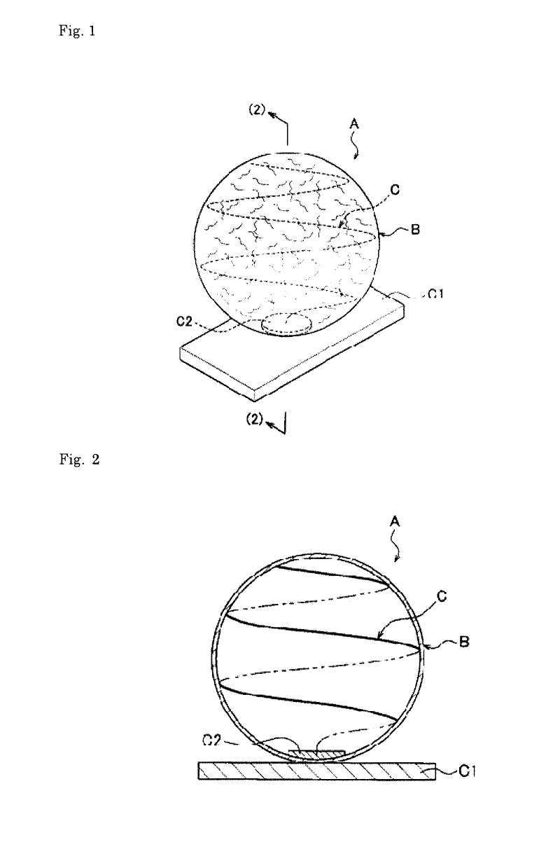

FIG. 1 and FIG. 2 show a figurine A as an example of a flat cocoon product. Note that the form of the figurine A explained below is merely an example of an embodiment of the flat cocoon product in the present invention and it is not limited to the form according to the present invention.

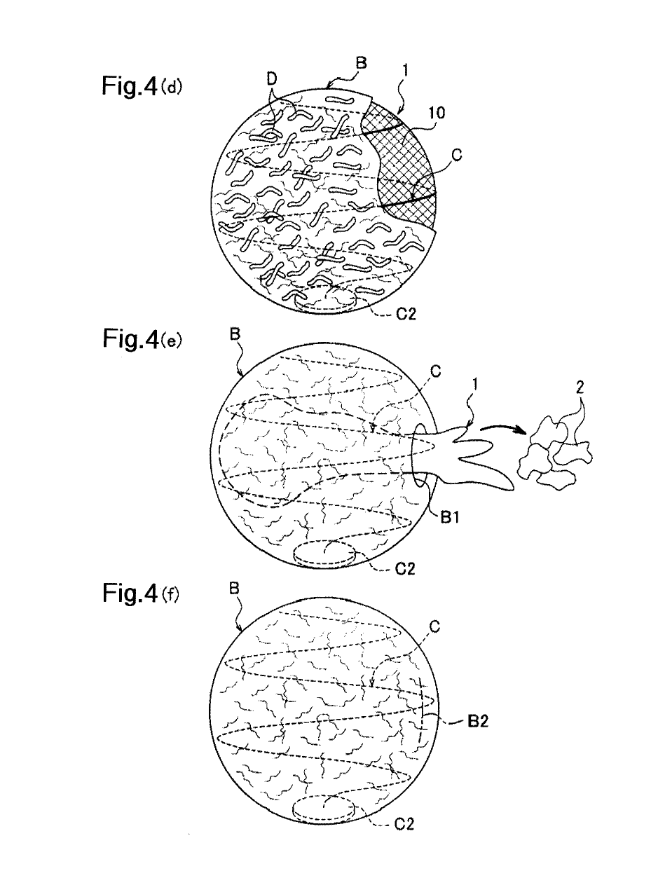

The figurine A is made from a flat cocoon B which is molded into a spherical body by causing silkworms D to crawl on the outer surface of the spherical three-dimensional molding die 1 and spin a cocoon (refer to FIG. 4(d)). With this figurine A a decorative member C is spirally disposed on the inner face thereof and the decorative member C is visible from the outer surface of the figurine A.

The decorative member C is, for instance, an EL fibre or an EL cord which emits light from the entire peripheral face without generating heat during the emission, is deformable and lightweight and, by using this kind of decorative member C, it is possible to prevent the figurine A from affecting the flat cocoon B during the emission as well as contribute to the weight saving of the figurine A.

Moreover, power is supplied to the decorative member C via a wireless power supply system and the decorative member C is provided with a power receiving unit C2 which receives power by being placed opposite to a power transmitting unit C1 of a wireless power supply. The power receiving unit C2 is disposed on the inner face of the figurine A and by mounting the figurine A in a state where the power receiving unit C2 is placed opposite to the power transmitting unit C1, it is possible to receive power from the power transmitting unit C1 and supply power to the decorative member C.

As a result of adopting this kind of decorative member C to which power is supplied via the wireless power supply system, a power cable for supplying power to the decorative member C is no longer required and the flexibility in selecting the setting place of the figurine A can be increased.

The power receiving unit C2 may also be a type comprising a rechargeable battery (not shown) and the flexibility in selecting the setting place of the figurine A can be further increased by adopting this kind of power receiving unit C2.

With this figurine A, since the light emitted from the spirally arranged decorative member C is visible from the outer surface, the figurine A has high designability and is also lightweight since it does not comprise a molding die.

Moreover, since the wireless power supply system is adopted as the method for supplying power to the decorative member C, the figurine A can be set at various locations and the existence of items (power cable, plug, etc.) which will impair the design or atmosphere can be eliminated during the setting of the figurine A and, therefore, it is possible to prevent the impairment of the design of the figurine A itself and the peripheral design upon setting the figurine A.

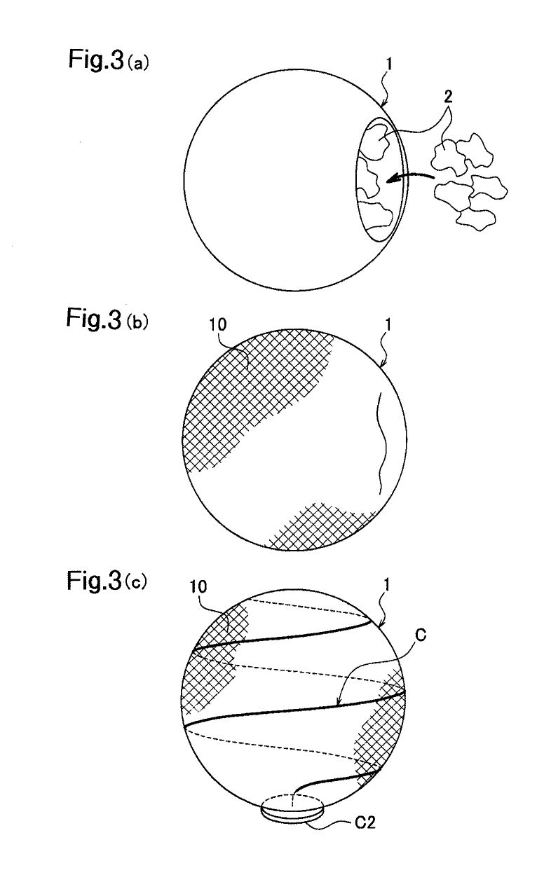

An example of the method of producing the foregoing figurine A is now explained with reference to FIGS. 3(a) to 3(c) and FIGS. 4(d) to 4(f). The form of the three-dimensional molding die 1 and the form of the decorative member C used in the production method explained below are merely examples of an embodiment of the method of producing a flat cocoon product in the present invention and are not intended to limit the present invention.

First step (FIG. 3(a), three-dimensional molding die expansion step): The three-dimensional molding die 1 in a contracted state is expanded.

The three-dimensional molding die 1 used here is of a flexible and thin bag-shaped molding die made from a paper material or a synthetic resin material and the three-dimensional molding die 1 is squashed from the outside or pressed outward from the inside so as to obtain the intended outer shape (spherical body) of the figurine A while placing a padding 2 made of crumpled thin paper materials into the three-dimensional molding die 1.

Second step (FIG. 3(b), net placement step): A net 10 which is to become the footing of the silkworms D is placed on the outer surface of the expanded three-dimensional molding die 1.

The net 10 used here is a net made of a fine net yarn and small mesh and it is possible to cause the silkworms D to crawl on the spherical body three-dimensional molding die 1 without falling off and prevent the fiber spun by the silkworms D from becoming entangled with the net yarn by covering the entire outer surface of the three-dimensional molding die 1 with the net 10 and bonding the net 10 thereto so that there is no gap between the net yarn and the outer surface of the three-dimensional molding die 1.

In other words, since the silkworms D can easily crawl on the outer face of the three-dimensional molding die 1 and it is possible to cause the silkworms D to spin a cocoon on the outer surface of the three-dimensional molding die 1 including the net 10 while preventing the fiber spun by the silkworms D from becoming entangled with the net yarn by placing the net 10 on the outer surface of the three-dimensional molding die 1, the three-dimensional molding die 1 including the net 10 can be removed from the molded flat cocoon B while preventing the fiber from becoming entangled and coming off.

Note that if the three-dimensional molding die 1 is made from a material which can ensure the footing of the silkworms D and prevent the silkworms D from slipping while they are crawling, the second step of placing the net 10 may be omitted.

Third step (FIG. 3(c), decorative member arrangement step): The decorative member C is spirally arranged on and wrapped around the outer surface of the three-dimensional molding die 1.

Here, it is possible to prevent the fiber spun by the silkworms D from becoming entangled with the decorative member C or the power receiving unit C2 by bonding the decorative member C and the power receiving unit C2 so that there is no gap relative to the outer surface of the three-dimensional molding die 1.

When a deformable member such as an EL fibre or an EL cord is used as the decorative member C, the decorative member C may be freely set in a shape other than the illustrated spiral shape. Moreover, a decorative member C that is different from the EL fibre or the EL cord may be additionally provided.

Fourth step (FIG. 4(d), spinning step): A plurality of silkworms D is caused to crawl on the outer surface of the three-dimensional molding die 1 and spin a cocoon.

As a result of causing the silkworms D to spin a cocoon as described above, it is possible to cause the silkworms D to spin a non-woven flat cocoon B along the outer surface of the three-dimensional molding die 1 rather than spinning a cocoon ball.

Moreover, due to the individual characteristics of the plurality of silkworms D, the crawling speed, crawling direction, spinning amount, spinning direction and the like will differ. Thus, the flat cocoon B will have portions with much fiber or portions with less fiber and various fiber patterns which cannot be predicted due to the shapes of these portions will arise and these fiber patterns can be visually recognized as the pattern of the figurine A.

The fiber patterns described above will enable the flat cocoon B to have portions having different transmission amounts of light from the decorative member C based on portions with much fiber or portions with less fiber and the shapes of these portions. In addition, as a result of providing these portions having different transmission amounts of light, it is possible to create portions of different brightness on the surface of the figurine A and such brightness can be visually recognized as the pattern of the figurine A.

The thickness of the flat cocoon B can be adjusted by lengthening or shortening the spinning time of the silkworms D and the thickness of the flat cocoon B can thereby be arbitrarily adjusted to a thickness corresponding to the various flat cocoon products.

It is possible to prevent the flat cocoon B from being contaminated with droppings by causing the silkworms D to sufficiently relieve their droppings prior to crawling on the outer surface of the three-dimensional molding die 1 and preventing the silkworms D from relieving their droppings while they are spinning a cocoon. Moreover, even if the flat cocoon B is contaminated with droppings, the flat cocoon B can be washed with water or a detergent or the like since it is non-woven.

Fifth step (FIG. 4(e), three-dimensional molding die extraction step): The three-dimensional molding die 1 is contracted and removed from the molded flat cocoon B.

Upon removing the three-dimensional molding die 1, a part of the flat cocoon 13 is cut to form an extraction hole B1 of the three-dimensional molding die 1 and the padding 2 that was filled in the three-dimensional molding die 1 can be removed from the extraction hole B1 and the three-dimensional molding die 1 can also be contracted and extracted.

The extraction hole B1 is preferably formed, as small as possible, in a size which enables the removal of the padding 2 and the extraction of the contracted three-dimensional molding die 1 so that the closing portion B2 will be unnoticeable upon closing the extraction hole B1 in the sixth step described below.

Sixth step (FIG. 4(f), extraction hole closing step): After the completion of the fifth step, the extraction hole B1 is closed via sewing or bonding.

Here, the closing portion B2 of the extraction hole B1 is preferably sewn or bonded so that it becomes unnoticeable. When the closing portion B2 is to be sewn or bonded as described above, preferably, a non-woven patch (not shown) made from the same material as the flat cocoon B is used and the outer peripheral edge of the patch and the fiber around the edge of the extraction hole B1 are loosened and mutually intertwined so that the closing portion 132 becomes unnoticeable.

It is possible to produce the figurine A having the decorative member C which emits light on its inner face without a molding die based on the first step to the sixth step described above.

It is thereby possible to enable weight saving and flexible designability of the three-dimensional figurine A, as well as the reliable spinning of the flat cocoon B by silkworms D.

Accordingly, it is possible to provide a three-dimensional figurine A which is lightweight, has a satisfactory design and yields a highly uniform flat cocoon B throughout the entire area thereof.

Moreover, it is possible to cause the decorative member C which emits light from the inside of the figurine A and be visible from the outer surface by mounting the figurine A in a state where the power receiving unit C2 is placed opposite to the power transmitting unit C1, since the decorative member C comprises the power receiving unit C2 for use in wireless power supply.

Moreover, the three-dimensional molding die 1 can be formed in various shapes, since the three-dimensional molding die 1 is of a flexible and thin bag-shaped molding die made from a paper material or a synthetic resin material and the three-dimensional molding die 1 can be formed into the intended shape by placing and deforming a padding 2 made from crumpled thin paper materials in the three-dimensional molding die 1.

The outer surface of the flat cocoon B after the completion of the fourth step or the outer surface of the figurine A after the completion of the sixth step may be colored or decorated with drawings or texts. Here, the third step of arranging the decorative member C may be omitted and the figurine A may be configured not to include the decorative member C.

Moreover, when the flat cocoon product is to be used as a shade of lighting equipment (light bulb or, the like), the sixth step may be omitted and the extraction hole B1 that was cut in the fifth step may be used as is as the insertion and removal opening of the lighting equipment or the area around the extraction hole B1 may be cut to form the insertion and removal opening of the lighting equipment.

1. A method of producing a flat cocoon product comprising:
expanding an expandable/contractible three-dimensional molding die;
molding a flat cocoon by causing silkworms to crawl on an outer surface of the three-dimensional molding die and spin a cocoon; and
contracting the three-dimensional molding die after the molding of the flat cocoon and extracting the contracted three-dimensional molding die from the flat cocoon.

2. The method of producing a flat cocoon product according to claim 1, further comprising:
arranging a decorative member on the surface of the three-dimensional molding die in an expanded state, wherein
the contracted three-dimensional molding die is extracted in a state where the decorative member is left on an inner face of the molded flat cocoon.

3. A flat cocoon product produced by the method of producing a flat cocoon product according to claim 1.

4. A flat cocoon product produced by the method of producing a flat cocoon product according to claim 2.
